(19) United States
(12) Patent Application Publication
(10) Pub. No.: US 2024/0325771 A1
(43) Pub. Date: Oct. 3, 2024

(54) Oscillation Patterns for Oncomagnetic Treatment

(71) Applicant: THE METHODIST HOSPITAL, Houston, TX (US)

(72) Inventors: Santosh A. Helekar, Sugar Land, TX (US); David Stuart Baskin, Houston, TX (US); Martyn Alun Shape, Houston, TX (US); Kumar Pichumani, Sugar Land, TX (US)

(21) Appl. No.: 18/699,264

(22) PCT Filed: Oct. 5, 2022
(86) PCT No.: PCT/US22/77635

§ 371 Date: Apr. 5, 2024

Related U.S. Application Data
(60) Provisional application No. 63/262,143, filed on Oct. 5, 2021.
(60) Provisional application No. 63/293,043, filed on Dec. 22, 2021.

(51) Int. Cl. A61N 2/00; A61N 2/12

(52) U.S. Cl. CPC A61N 2/004; A61N 2/12

(57) ABSTRACT

A method for disrupting mitochondrial function in cells includes causing one or more magnets to oscillate along at least a first axis of rotation and a second axis of rotation substantially orthogonal to the first axis of rotation, so as to generate an oscillating magnetic field. To this end, the method includes selecting the first axis of rotation, applying an oscillation pattern along the first axis of rotation, the oscillating including a ramp-up period for an oscillation frequency, a ramp-down period for the oscillation frequency, and an inactive period following the ramp-down period, selecting the second axis of rotation, and applying the oscillation pattern along the second axis of rotation. The method further includes applying the oscillating magnetic field to a tissue comprising cancer cells to trigger apoptosis in the cancer cells.

### FIELD OF THE DISCLOSURE

This disclosure generally relates to cancer treatment methods and apparatus and, more particularly, to non-invasive cancer treatments utilizing oscillating magnetic fields.

### BACKGROUND

Cancer is one of the biggest health problems facing modern society, and improvements in aggressive treatments for some forms of cancer, including glioblastoma (GBM) remain low. Common forms of cancer treatment include chemotherapy and radiotherapy which can be devastating to the patient's body, causing severe physical and mental trauma. The heavy toll on patients can result in a patient deciding not to complete the suggested chemotherapy or radiation treatment cycles. Thus, there is a need for a cancer treatment approach with better life-expectancy outcome and less toxicity.

Recently, applying alternating electric fields (AEFs) to the scalp, a treatment called Tumor Treating Field (TTF) therapy or Optune® therapy, has shown therapeutic benefit in patients with GBM. TTF therapy has been approved by the U.S. Food and Drug Administration as monotherapy for GBM and in combination with other therapies for newly diagnosed GBM. TTF therapy is commonly administered to patients by attaching electrodes to a patient's head, which requires shaving the patient's head at electrode sites. Moreover, electrodes often cause lesions on the skin, rashes, other dermatological adverse effects, and in some cases even cause burns due to applied electrical currents.

Cancer cells are known to possess high levels of reactive oxygen species (ROS). ROS levels in cells play diverse roles in normal cellular processes such as developmental cell proliferation and differentiation, programmed cell death, cell motility, immune defense mechanisms, inflammation, and neuronal activity and plasticity. They are also involved in cancer cell proliferation and tissue invasion, on the one hand, and cellular aging and neurodegeneration, as mediators of oxidative stress, on the other. Cancer cells are known to possess high levels of ROS because of increased oxidative metabolism and dysfunctional mitochondria. Previous studies have shown that abnormally high levels of ROS cause apoptosis.

### SUMMARY

Generally speaking, the system or device of this disclosure applies oscillating magnetic fields (sOMF) to a tissue in a manner that causes cell death (e.g., apoptosis) in cancer cells (e.g., GBM). The system can include one or more magnetic assemblies and controlling hardware, such as one or more processors and non-transitory computer-readable medium storing instructions executable by the controlling hardware. Each magnetic assembly, also referred to below as an “oncoscillator,” can include a motor configured to impart rotational motion to a magnet, diametrically magnetized relative to the axis of rotation, according to a particular pattern. The system is configured to generate, sequentially or simultaneously and relative to certain tissue, magnetic fields that oscillate around orthogonal axes. Depending on the implementation, the system includes multiple magnetic assemblies disposed around the issue or a single magnetic assembly configured to change the axis of rotation, e.g., by rotating the axis around which the corresponding magnet rotates by approximately 90 degrees in one plane, and/or by approximately 90 degrees in another plane.

Certain specific amplitude, frequency, and timing parameters of rapidly rotated magnetic field produce a maximal rise in superoxide in glioma cells. In particular, a particular range of parameters of dynamic magnetic fields show peak efficacy in inducing ROS and preventing formation of cancer cell colonies. Both oscillations of the field and rotations of the axis together are relevant to efficacy of the treatment, in an example implementation.

According to some example configurations, the system performs stimulation of brain tissue intermittently for 1-4 hours at a time, 2 or 3 times a day. The system either rotates the axis of a magnetic assembly or uses multiple magnetic assemblies, so that the axis around which the magnet rotates is orthogonal relative to the axis of another magnetic assembly, or orthogonal relative to both axes of two other magnetic assemblies. The system can activate the magnetic assemblies sequentially or simultaneously; thus, the system can expose tissue to orthogonal sOMF sequentially or simultaneously. Each of the magnet assemblies generates an oscillating magnetic field. The system creates an effective magnetic field in the tissue of strength in the range of 1-200 mT. At each magnetic assembly, the system generates a pulse train of duration between 250 and 500 milliseconds, with an OFF period between stimulus pulse trains of 250 to 2,750 milliseconds. The system can ramp up a pulse train ramps up from zero to a peak frequency over a period of 50 to 100 millisecond, ramps down the pulse train to a frequency of less than ˜50 Hertz over a period of 50-200 milliseconds. The peak frequency may be in the range of 100-300 Hertz.

The system, which can be referred to as an “oncomagnetic” system or device, operates in a non-invasive manner. In particular, the system can apply sOMFs without directly contacting the subject's scalp when used treat to a brain tumor, or the subject's skin when used with other parts of the body.

In operation, the system raises reactive oxygen species (ROS) levels in oxidatively stressed cancer cells to cause macromolecular damage and cell death, as part of anticancer treatment strategy. The system consistently raises ROS to levels that are cytotoxic to patient-derived glioblastoma cells, but not normal brain cells.

Because the system can include stimulators equipped with permanent magnets and an electric motor configured to communicate oscillating motion (e.g., rotation, translational oscillation) to the permanent magnets, these techniques advantageously do not require passing a large current through coils or solenoids, for example. In some implementations, the stimulators are miniaturized and are referred to as “microstimulators.” When the system includes multiple stimulators, the controlling hardware can cause the stimulators to operate at different times, at different frequencies, at different pulse rates, etc. to define a particular stimulation pattern, which can be subject-specific.

One embodiment of these techniques is a method for disrupting mitochondrial function in cells. The method includes causing, by controlling hardware, a one or more magnets along (i) a first axis and a (ii) a second axis substantially orthogonal to the first axis, to oscillate so as to generate an oscillating magnetic field, and applying the oscillating magnetic field to a tissue comprising cells with mitochondrial impairment to trigger apoptosis in the cells with mitochondrial impairment. In a related embodiments, causing the one or more magnets to oscillate further comprises causing the one or more magnets to oscillate along a third axis substantially orthogonal to the first axis and the second axis.

### BRIEF DESCRIPTION OF THE DRAWINGS

FIG. 1 is a block diagram of a system for disrupting mitochondrial function in certain cells by applying an oscillating magnetic field, according to one example implementation.

FIG. 2 schematically illustrates a magnetic assembly that can operate in the system of FIG. 1.

FIG. 3 illustrates example oscillatory magnetic field output generated by the device of FIG. 2.

FIG. 4 illustrates sequential activation of different magnetic assemblies along different axes.

FIG. 5 schematically illustrates placement of magnetic assemblies on a spherical surface.

FIG. 6 is a flow diagram of an example method of applying oscillating magnetic fields to a tissue for the purposes of oncomagnetic treatment, which can be implemented in the system or oncomagnetic device of FIG. 1.

FIG. 7 is a schematic diagram of the cell culture sOMF stimulation setup used in the laboratory, with a blown-up view of a cell culture dish placed above each oncoscillator displayed on the right side.

FIG. 8A illustrates a scheme for stimulation of cultured cells with static magnetic field of one stationary magnet (left section), three stationary magnets in three orthogonal axis (middle section), and oscillating magnetic field of one spinning magnet (right section).

FIG. 8B illustrates fluorescence intensity quantitation of hydroethidine in GBM (GBM115) cells.

FIG. 8C illustrates fluorescence intensity quantitation of hydroethidine in DIPG cells using microscope images; in particular, for each of the three periods: a Ctrl element corresponds to the unstimulated case, an 3DSt element corresponds to stimulation with the magnetic field of three stationary magnets in orthogonal axis, a 1Dst element corresponds to one stationary magnet, and a 1Dsp element corresponds to one spinning magnet.

FIG. 8D illustrates fluorescence intensity quantitation of hydroethidine in GBM (GBM115) cells.

FIG. 8E illustrates fluorescence intensity quantitation of hydroethidine in DIPG cells using microscope images; in particular: for each of the three periods, a Cont element corresponds to a continuously spinning magnet, an Int element corresponds to a magnet spinning intermittently for 250 ms (Tₒₙ) and stop for 250 ms (T_{off}), and a Ctrl element corresponds to control. The scatter with bar represents average normalized fluorescence from three independent experiments with each data point shown as dot (n=24). Error bars show SEM.

FIGS. 9A and 9B are bar graphs with scatter showing cellular ROS levels in GBM115 cells (FIG. 9A) and DIPG cells (FIG. 9B), respectively, normalized to pre-stimulation control baseline during (2 h and 4 h) and 2 h post-stimulation with the Helmholtz coil and a single oncoscillator with magnet rotating in a 2-dimensional plane. In these cases, the stimulation was continuous for 4 h at ˜137 Hz PPA. Error bars show SEM.

FIG. 10A illustrates a scheme for stimulation of cultured cells with sOMF of one spinning magnet (1Dsp) at 250 ms Tₒₙ and 250 ms T_{off} or three spinning magnets (3DSp) with sequentially turning on and off for 250 ms Tₒₙ and 250 ms T_{off}.

FIGS. 10B and 10C illustrate fluorescence intensity quantitation of hydroethidine in GBM (GBM115) cells or DIPG cells, respectively, using microscope images. a Ctrl element corresponds to the unstimulated case, an 3DSt element corresponds to stimulation with the magnetic field of three stationary magnets in orthogonal axis, a 1Dst element corresponds to one stationary magnet, and a 1Dsp element corresponds to one spinning magnet. Scatter with bar represents average normalized fluorescence intensity from three independent experiments with each data point shown as dot (n=24). Error bars show SEM.

FIG. 11A depicts the positions of the cell culture dish relative to the oncoscillator to obtain three different values of peak-to-peak amplitude (PPA) or magnetic field strength, in particular: the bar graph with scatter plots show the effect of variations in PPA (FIGS. 11B and 11C) and peak frequency (PF) (FIGS. 11D and 5E). Data points and asterisks are as denoted in FIG. 1 (n=24 and n=40, respectively).

FIG. 12A illustrates a scheme for sOMF stimulation of cultured cells with a constant Tₒₙ of 250 ms and T_{off} of 250 ms, 750 ms and 2750 ms.

FIGS. 12B and 12C illustrate fluorescence intensity quantitation of hydroethidine in GBM (GBM115) cells and DIPG cells, respectively, using microscope images. Scatter with bar represents average normalized fluorescence intensity from three independent experiments with each data point shown as dot (n=24). Error bars show SEM.

FIGS. 13A and 13B illustrate fluorescence intensity quantitation of hydroethidine in GBM (GBM115) cells and DIPG cells, respectively, using microscope images. Scatter with bar represents average normalized fluorescence intensity from three independent experiments with each data point shown as dot (n=32). Data points and asterisks are as denoted above. Error bars show SEM.

FIGS. 14A and 14B are scatter with bar graphs that show survival fraction in clonogenic cell survival assay for GBM (GBM115) and DIPG cells, respectively, from independent experiments with each data point shown as dot (n=12). Error bars show SEM. Representative images of caspase-3 activity increase 12 hours after 4-h sOMF exposure in GBM and DIPG cells.

FIGS. 15A and 15B illustrate fluorescence intensity measurements of hydroethidine stained GBM (BT 115) and DIPG cells, respectively, exposed to different magnetic field strengths (in mT). Scatter with bar graph show mean of n=4 and error bar show SEM.

FIG. 16A represents the influence of magnetic field on electron spins.

FIG. 16B illustrates the hypothetical effect of magnetic field as a function of its strength.

FIG. 17 schematically illustrates the interaction between the system of FIG. 1 and mitochondrial function of a cell.

### DETAILED DESCRIPTION

### Overview

Generally speaking, stimulation by electromagnetic field (EMF) generating devices can raise ROS levels in cancer cells and thereby induce cell death of malignant tumor cells in vitro. It is difficult however to develop device that both safe and efficacious. Further, it is difficult to ascertain the precise range of physical parameters of EMF that produce a potentially therapeutic increase in ROS levels.

The non-invasive EMF device of this disclosure can address these limitations because its stimulus parameters can be better and more precisely controlled and targeted. The device generates oscillating magnetic fields (sOMF) by rapidly rotating strong neodymium permanent magnet. Experimental data suggests that this device, which can be referred to as an “oncomagnetic” device, substantially and consistently raises ROS in patient derived glioblastoma (GBM) to levels that are selectively cytotoxic to these cells, while sparing normal cells. Evidence also has been obtained for the safety and efficacy of the device in mice implanted with orthotopic GBM xenografts and in a patient with recurrent GBM with no standard of care treatment options.

The sOMF-induced increase in ROS is likely due to perturbation of the electron transfer process in the mitochondrial electron transport chain (ETC). This Magnetic Electron Perturbation (MEP) hypothesis is proposed because of the known effect of weak (<1 mT) and intermediate range (1 mT-10 mT) magnetic fields on mixing of spins of unpaired electrons of free radical intermediates during the spin-correlated electron pairing process termed as the radical pair mechanism (RPM).

The device is programmable and allows precision control of all physical parameters of sOMF exposure (stimulation) both in vitro and in vivo, such as strength of the magnetic field, frequency of field oscillations, angles of rotation of the field axes, on and off intervals of intermittent stimulation, and duration and frequency of stimulation. The device therefore allows one to determine the precise range of parameters that achieve consistent optimum increase in ROS. Furthermore, it also allows one to more fully explore the relationship between aspects of the field generated by the device and its interaction with the physical characteristics of RPM as it manifests in the mitochondrial ETC protein complexes. For example, because these complexes have transmembrane orientations that are fixed in three dimensions over the time frame of sOMF pulse trains, one can test the possibility that changes of the magnetic field axis angle in three dimensions as can be done only with a rotating magnet device is more effective at inducing ROS, as opposed to static field distributed along one axis.

The device can be configured, and the corresponding procedure can be parameterized, based on the effects of varying each of the physical parameters that define the sOMF produced by the active components of the device called “oncoscillators” in patient derived GBM and diffuse intrinsic pontine glioma (DIPG) cells. The efficacy of a range of stimulus strengths, frequencies, and on-off periods in inducing ROS has been tested. Further, it has been investigated whether static magnetic fields with orthogonally oriented axes in three dimensions and sOMF produced by a non-rotating Helmholtz coil can induce ROS to the same extent as a rotating magnetic field. This was to differentiate whether changing angles of the magnetic field axes or field oscillations produced by spinning magnets are critical for the ROS-inducing effect. The latter could be important, in addition, to establishing resonance with the periodic and cyclic nature of the electron transfer process. Still further, anticancer efficacy of the optimized ROS-inducing stimulation parameters in clonogenic cell survival and caspase 3 activation assays in GBM and DIPG cells has been tested.

### Overview of the System

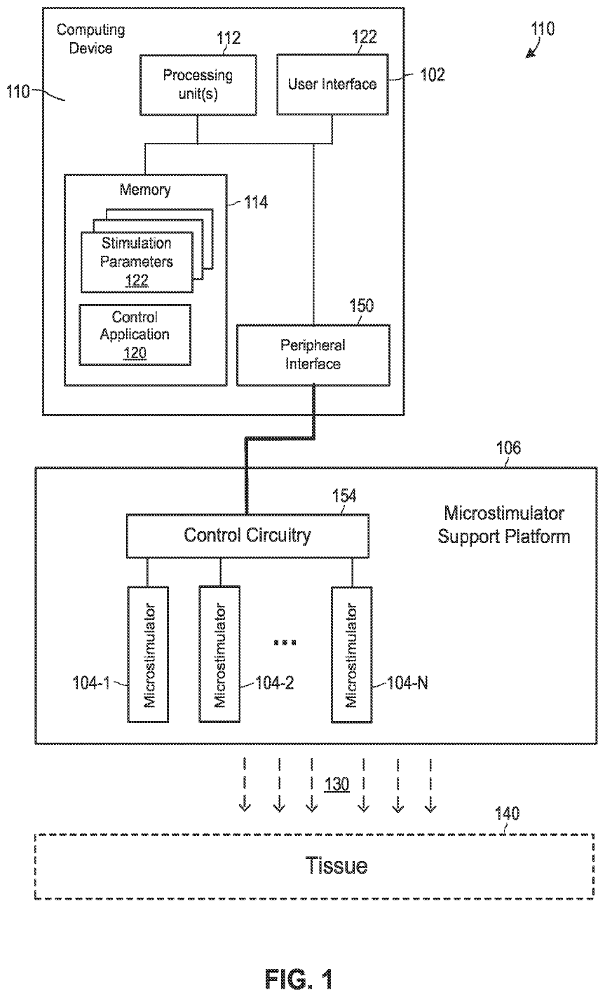

FIG. 1 illustrates a system of this disclosure, which applies oscillating magnetic fields, and more particularly, rapidly changing magnetic fields, to tissue so as to target cancer cells. Generally speaking, cancer cells have altered bioenergetics, mitochondrial function, and a reduced number of mitochondria due to uncontrolled cell division.

The system of this disclosure uses electromagnetic fields (EMF) to modulate cellular metabolism. Long exposures to repeated EMF pulses, radiating radiofrequency waves, or non-radiating local field oscillations have varying degrees of efficacy against cancer cells in culture. Cancer cells are oxidatively stressed, compared to normal cells of the same tissue, and have high levels of reactive oxygen species (ROS) which, if accentuated further, leads to cancer cell apoptosis. The application of EMF pulses on cells causes an increase in the intracellular levels of ROS leading to cell apoptosis. Disclosed herein are methods and systems for generating an oscillating magnetic field (OMF) for in vitro and in vivo treatment of tumors and cancerous tissue by inducing the generation of ROS and causing cancer cell apoptosis.

As described herein, “magnetic stimulators,” “oncoscillators,” or “microstimulators” generate OMFs through the rotation of permanent magnets at high speeds. The microstimulators are modified to produce patterns of magnetic field oscillations that cause selective apoptosis of cultured GBM cells, without causing apoptosis in normal astrocytes. The lack of lethality in normal cells is due to an abundance of mitochondria, absent or reduced oxidative stress, and much lower demand for ATP compared to rapidly dividing malignant cells. In fact, repetitive transcranial magnetic stimulation has shown decreased apoptosis in non-cancerous cells. By applying OMFs at certain ranges of frequencies applied in defined pulsed patterns, the system causes the disruption of electron flow in the mitochondrial electron transfer chain (ETC), in turn causing the generation of ROS. The ROS cause the opening of the mitochondrial permeability transition pore (MPTP), resulting in mitochondrial membrane depolarization and extrusion of cytochrome C, and triggering caspase-dependent apoptosis or apoptosis by an alternate mechanism in the cancer cell. The microstimulators may also be referred to herein as oncoscillators due to the tumor-tissue selective nature of the treatment methods and systems described herein. In various embodiments, the systems can include microstimulator probes, wearable apparatus with multiple oncoscillators, and fixtures for surrounding a patient or body of tissue for treatment.

The OMF therapy methods and systems described do not have the limitations of chemotherapeutic agents such as the need for adequate blood supply to all parts of the malignant tumor, the ability to penetrate the blood brain barrier in case of brain cancers, a high enough bioavailability, a favorably tuned pharmacokinetic profile, a sufficiently large therapeutic index, etc. In addition, a variety of OMF treatment delivery mechanisms are available with great flexibility and versatility to have a substantial impact on all types of primary and metastatic solid neoplasms, and possibly systemic malignancies as well. Additionally, it is possible to administer OMF treatment sessions as only one to three hours of application a day, whereas TTF therapy requires 18 to 20 hours of treatment each day. The proposed methods and systems for OMF therapy cost much less than TTF therapy. The specific OMF frequencies and amplitudes selectively kill cancer cells in any stage of the cell cycle and do not depend on cell division, depend on being in a mitotic state, or depend on any other state. In fact, the methods and systems disclosed can selectively kill cancer cells in the GO phase of the cell cycle. The method and systems for oncomagnetic therapy described herein can be drug-free, ionizing radiation-free, and non-invasive, or oncomagnetic therapy may be performed in conjunction with other forms of therapy such as with chemotherapy, other forms of radiative therapy, with drugs and prescriptions, etc.

According to at least some of the techniques of this disclosure, a system generates oscillating magnetic fields to induce alteration of electron flow in a tissue. However, it is believed that at least some of these techniques also can be used with AEF techniques to eliminate the need to directly apply electrodes to the patient's skin, for example. More particularly, it may be possible to use oscillating magnets to generate AEFs with properties similar to those used in TTF therapy.

### An Example System for Disrupting Mitochondrial Function in Cells

FIG. 1 illustrates an example implementation system 100 for disrupting mitochondrial function in certain cells by applying a rapidly changing magnetic field. The system 100 includes a computing device 102 that controls magnetic stimulators 104-1, 104-2, . . . 104-N mounted on a stimulator support platform 106, which can be a probe, helmet, a brace, a belt, mechanical frame, room, bed, cubicle, etc. Because the stimulators 104 can be miniaturized to be, for example, 500 millimeters, 1 cm, 2 cm, etc. along the longest dimension, the magnetic stimulators 104 are referred to below as microstimulators 104. Further, due to the particular application of the magnetic fields discussed herein, the magnetic stimulators 104 can be referred to as “oncoscillators.” The computing device 102 includes controlling hardware 110 that can include one or more processing units 112 coupled to a non-transitory computer-readable memory 114 storing a control application 120 and stimulation parameters 122.

In operation, the controlling hardware 110 causes the microstimulators 104 to generate an oscillating magnetic field 130 and, using the stimulator support platform 106, apply the oscillating magnetic field 130 to a tissue 140. The microstimulators 104 generate the oscillating magnetic field 130 in a manner that causes disruption of mitochondrial function in cells of the tissue 140. As discussed in more detail below, the oscillating magnetic field 130 disrupts electron flow in the cells, such that apoptosis is triggered in the cancer cells, but no apoptosis is triggered in the healthy cells of the tissue 140.

In addition to being capable of altering electron flow in cell tissue without directly contacting the subject's scalp or skin on other parts of the body, the system 100 provides an additional advantage of being configurable for a variety of different solid cancers. As discussed below, various stimulation parameters and stimulation parameters can generate different configurations of OMF, which can be tailored for a specific cell type and/or specific subject to implement a personalized treatment protocol. Further, the system has the advantage of an imperceptible sham treatment capability to serve as a placebo control in double-blind trials. In particular, high-field strength magnets in the microstimulators discussed below can be replaced with demagnetized magnets. Because a human subject cannot sense the magnetic field, research subject and investigators cannot distinguish between an operational instance of the system 100 and a sham stimulation system by inspection or during a treatment session.

The computing device 102 can be a general-purposed computing device such as a desktop computer, a laptop computer, a table computer, a smartphone, a wearable device such as a smartwatch, etc. The one or more processing units 112 in these implementations can be central processing units (CPUs), and the memory 114 can include persistent components (e.g., a flash drive, a disk) as well as non-persistent components (e.g., Random Access Memory (RAM)). In other implementations, the computing device 102 is a special-purpose medical device configured specifically to control the one or more microstimulators 104. The processing units 112 in this case can include a field programmable gate array (FPGA), an application-specific integrated circuit (ASIC), or any other type of special-purpose hardware. Further, the controlling hardware 110 in some implementations is embedded in the stimulator support platform 106.

The control application 120 can and one or more sets of stimulation parameters 122 which can specify, for a certain stimulation session, at least some of: the duration of the session, pulse width (length) of stimulus pulses, the duration of stimulus pulse intervals, and the frequency of oscillation of the magnets. The stimulation parameters 122 in some cases can define separate sets of values for different microstimulators 104-1, 104-2, . . . 104-N, to define a particular stimulation pattern. More particularly, the stimulation pattern can specify a certain time of activation and operational parameters (frequency, pulse parameters) for the microstimulator 104-1, specify a different time of activation and different operational parameters for the microstimulator 104-2, etc. These parameters in some cases can depend on the relative positioning of the microstimulators 104, so that for example a microstimulator 104-i and a microstimulator 104-j positioned at a certain distance and oriented at a certain angle relative to each other generate stimulation pulses with a certain phase offset, so as to generate an oscillating magnetic field with certain desired characteristics.

In the example of FIG. 1, the computing device 102 includes a peripheral interface 150 and a user interface 152, in addition to the one or more processors 112 and the memory 114. The peripheral interface 152 can be Serial Peripheral Interface (SPI), a Universal Serial Bus (USB), or any suitable wireless interface such as Wireless Personal Area Network (WPAN) interface (e.g., Bluetooth®), a Wireless Local Area Network (WLAN) interface (e.g., Wi-Fi®), etc. The computing device 102 can use the peripheral interface 150 to transmit commands to the microstimulators 104. More particularly, the computing device 102 in the example implementation of FIG. 1 provides commands to the microstimulators 104 via the peripheral interface 150 and a control circuitry 154 of the stimulator support platform 106.

The control circuitry 154 can be configured to receive commands for the individual microstimulators 104-1, 104-02, etc. and turn on or off the motors in the microstimulators 104-1, 104-02, etc., vary the speed of rotation or other type of oscillation in the motors, etc. The control circuitry 154 thus can operate as a demultiplexer. Further, as indicated above, in some implementations the controlling hardware 110 is embedded in the stimulator support platform 106, and thus the entire control functionality of the system 100 can be provided in the control circuitry 154. More generally, control functionality of the system 100, such as the control logic for activating the microstimulators 104 in accordance with various stimulation patterns, can be distributed between the computing device 102 and the control circuitry 154 in any suitable manner, including providing the entire control functionality entirely in the computing device 102 or entirely in the control circuitry 154.

The user interface 152 can include a touchscreen configured to receive input and display output or separate input (e.g., a keyboard, a pointing device) and output (e.g., a display) components. An operator can use the user interface 152 to provide commands to select the desired particular stimulation parameters 122 for a particular microstimulator or a stimulation pattern that includes multiple stimulation parameters 122 for the respective microstimulators. Further, the system 100 in some implementations can include one or more magnetic sensors such as microelectromechanical system (MEMS) sensors, and the user device 102 can provide readings from these magnetic sensors via the user interface 152.

The microstimulator platform 106 can be a harness, a helmet, a brace, etc. Further, the microstimulator platform 106 can be a part of a hospital bed, and the system 100 in some cases can apply stimulation to a sleeping subject. Still further, the microstimulator platform 106 in some implementations can be an intraoperative probe which a clinician can guide manually. In some implementations, the microstimulator platform 106 includes one or more magnetic sensors to provide sensor readings to the operator as discussed above and/or provide a feedback signal to the controlling hardware 110, so that the controlling hardware 110 can tune certain operational parameters to achieve the desired strength of the magnetic field.

The microstimulator platform 106 in some implementations can include a power storage device, such as a battery, to power the microstimulators 104. The battery can be for example a Tenergy NiMH 9.6V, 5,000 mAh rechargeable battery. In other implementations, the computing device 102 can be configured to provide electric power to the microstimulator platform 106 via the peripheral interface 150.

The system 100 in various scenarios can generate stimulation sessions that are intermittent or continuous, ranging in duration from 1 minute to 20 hours for a given therapy session, depending on the amount of desired stimulation determined by a treatment plan. In various aspects, the oscillating magnetic field intermittently or continuously during of a period of between about 20-30 hours. The frequency of each oscillatory stimulus, and therefore the oscillatory motion of the motor, can range from 5 Hertz to 400 Hertz. In various aspects, the frequency of each oscillatory stimulus, and therefore the oscillatory motion of the motor is 30-300 Hertz. In various aspects, the frequency of each oscillatory stimulus, and therefore the oscillatory motion of the motor is about 300 Hertz. The microstimulators 104 can provide oscillatory stimulus as one or more pulses in a series. In some example implementations, the pulse lengths range from about 10 milliseconds to about 5 seconds, with inter-stimulus pulse intervals ranging from about 10 milliseconds to about 10 minutes, resulting in pulse train duty cycles ranging from 0.001% to 50%. The oscillatory frequency, timing, pulse duration, and inter-stimulus pulse interval of the oscillatory stimulus any of the microstimulators 104 provide may be kept the same, varied in groups, and/or varied independently for each microstimulators 104. The oscillatory stimulus pulses may vary in temporal length and inter-stimulus pulse interval from pulse to pulse as required to provide adequate field intensity to a target site or volume. The stimulus pulses may have amplitude envelopes whose shapes are square, Gaussian, sinusoidal, ramp, sawtooth, etc., to provide adequate field amplitude to a target site or volume. Additionally, the system 100 can ramp the oscillatory frequency up and/or down to maximum desired frequencies or between frequencies for a given therapeutic session.

In some implementations, the controlling hardware 110 can activate the microstimulators 104 one at a time, simultaneously, sequentially, in-pairs, in groups, or according to any combination thereof (e.g. sequentially in pairs, one group at a time). In fact, the controlling hardware 110 can activate any number of microstimulators 104 in any sequence able to deliver the desired OMF to a target site or volume.

The controlling hardware 110 in some cases can implement a beat frequency stimulation when generating OMF in a target site or volume. According to the beat frequency stimulation approach, the controlling hardware 110 causes the magnets in two microstimulators to oscillate at different frequencies so as to produce a beat frequency at a target site or volume. For example, the microstimulator 104-1 can oscillate at 320 Hz, while the microstimulator 104-2 can oscillate at 400 Hz. Due to electromagnetic interference, the microstimulator 104-1 and 104-2 generate a beat frequency of 80 Hz (which is the difference between the frequencies of 320 Hz and 400 Hz) at a target site or volume. The amplitude of the OMF at this beat frequency is twice the amplitude of the OMF the microstimulator 104-1 or 104-02 induces independently. The system 100 can implement beat frequency stimulation when high-amplitude low-frequency fields are required or desirable in treatment.

Due to the small dimensions of the magnets and motors in the microstimulators 104, as well as the ability to operate using a battery, the system 100 can be portable. Therefore, treatment may be performed at any time or location. Moreover, the control application120 can be installed on a smartphone, a tablet computer, or a portable computer device as discussed above.

As discussed above, the control application 120 can operate according to the stimulation parameters 122 and various stimulation patterns or preprogrammed therapy sessions. The control application 120 additionally can implement safety features such as limiting the amount of treatment a patient can receive daily, weekly, or monthly, limiting how often or when a patient can apply treatment (e.g., only on weekdays, only on a specific day of the week, only for a period of four weeks).

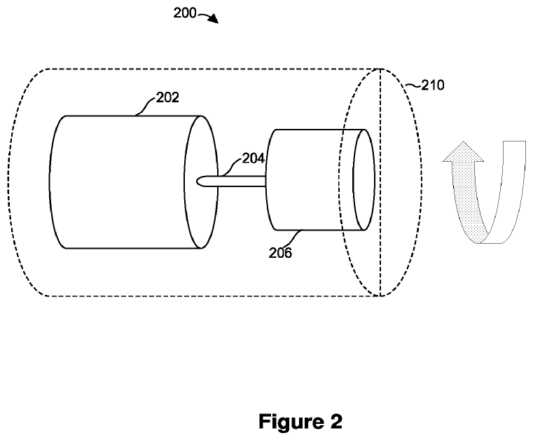

FIG. 2 illustrates an example magnetic assembly or oncoscillator 200 that can operate as an microstimulator 104 in the system of FIG. 1. The oncoscillator 200 includes an electric motor 202 and an a magnet 206 mounted on the axis 204. In this implementation, the magnet 206 is diametrically magnetized with a semicircular pole configuration, and the motor 202 imparts rotational motion to the magnet 206, in the direction indicated by the arrow (or the opposite direction). The motor 202 can be a high-speed motor capable of producing 21,000 rotations per minute (RPM) in a no-load mode. The oncoscillator 200 optionally may include a housing or sleeve 210, made from a material permeable to the magnetic field (e.g., plastic), so as to not affect the OMF generated by the oncoscillator 200.

The magnet 206 can have a cylindrical shape with a hole configured to receive the axis 204. The diameter of the magnet 206, suitable particularly for GBM treatment, approximately ¾ of an inch. The height of the cylinder defined by the magnet 206 is also ¾ of an inch, in an example implementation. The magnet 206 can be a relatively strong rare earth magnet with a strength above 1.2 Tesla and can be, for example, a Neodymium N42 magnet.

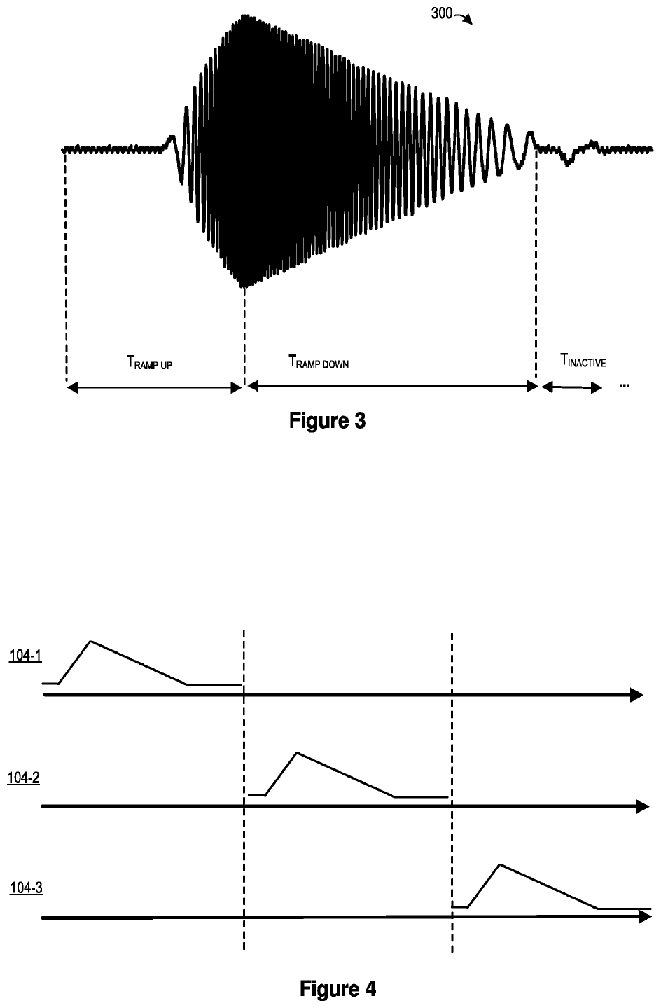

FIG. 3 illustrates example output 300 of the oncoscillator 200. To generate the output 300, the oncoscillator 200 ramps up the frequency from approximately 0 Hz to the peak frequency of approximately 150 Hz, over a period of T_{RAMP UP} (e.g., 50-100 ms). The oncoscillator 200 then can ramp down from the peak frequency to less than 50 Hz, or possibly down to 0 Hz, over a period of T_{RAMP DOWN} (e.g., 50-200 ms). In the example of FIG. 3, T_{RAMP DOWN} is significantly longer than T_{RAMP UP}. The oncoscillator 200 then can remain inactive for a period of T_{INACTIVE}, which can be between 250 and 2750 milliseconds, until a new cycle beginning again with a T_{RAMP UP} period.

More generally, the oncoscillator 200 can ramp up the frequency to the peak frequency of between 100 and 300 Hz, depending on the specific implementation.

As illustrated in FIG. 4, the oncomagnetic device of this disclose can sequentially activate multiple oncoscillators 104-1, 104-2, and 104-3 to affect the same tissue. Each of these oncoscillators can be implemented as illustrated in FIG. 2. The axis along which the oncoscillator 104-1 rotates the corresponding magnet is substantially orthogonal to the axis along which the oncoscillator 104-2 rotates the corresponding magnet, and each of the axes of the oncoscillators 104-1, 104-2 is orthogonal to the axis along which the oncoscillator 104-3 rotates the corresponding magnet. The pulse trains are illustrated in a simplified manner, but can be similar to the output 300 discussed above.

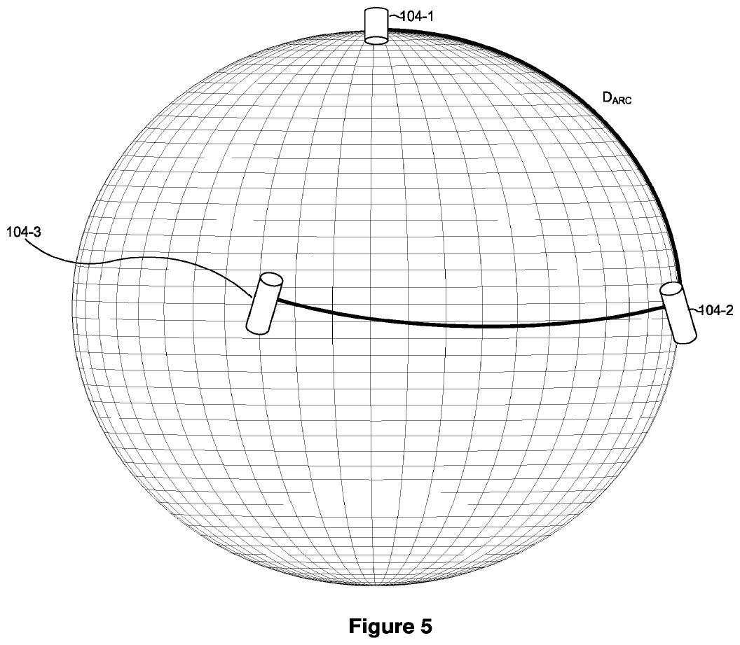

FIG. 5 illustrates example placement of three oncoscillators on the surface of a sphere, which approximates a patient's head, for the purposes of GBM treatment. Each of the oncoscillators 104-1, 104-2, and 104-3 can have an arcual separation of 10-12 cm relative to other oncoscillator. The magnetic flux penetrates to the depth of approximately 12 cm.

In an example implementation, the oncoscillators 104-1 and 104-2 are attached to the supporting head gear bilaterally in the front, and the oncoscillator 104-3 is attached to the back in the midline.

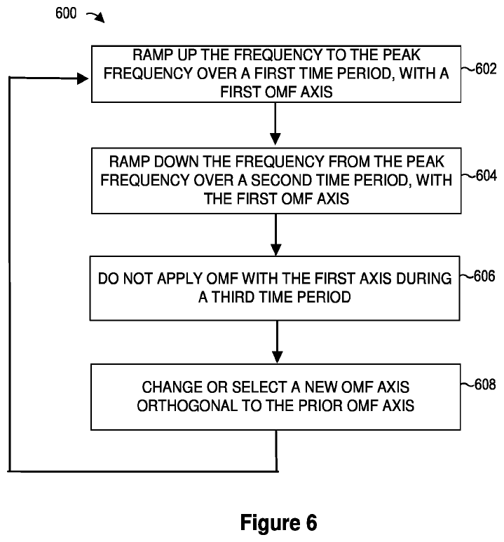

FIG. 6 illustrates an example method that can be implemented in the device of claim 1. At block 602, the oncomagnetic device ramps up the frequency to the peak frequency (e.g., 100-300 Hz) over a first period of time, e.g. 50-100 ms. At block 604, the oncomagnetic device ramps down the frequency from the peak frequency over a second period of time, e.g. 50-200 ms. At block 606, the oncomagnetic device does not apply OMF during the off period, e.g., 250-2750 ms. At block 608, the oncomagnetic device changes the axis along which the magnet rotates if a single magnet is used; or the oncomagnetic device selects another magnetic assembly with an axis orthogonal to the axis of the magnetic assembly active at block 602 and 604 (see FIG. 4). The flow then returns to block 602.

For further clarity, several experiments related to the oncomagnetic system of this disclosure are discussed below.

### Cell Lines and Reagents

At least some of the examples below relate to GBM115, which is a Temozolomide-resistant cell line that was derived from GBM patient tissue resected by DSB, and DIPG cell line was purchased from Sigma-Aldrich. Both cell lines were cultured in DMEM supplemented 10% fetal bovine serum, 11 mM Glucose and penicillin and streptomycin antibiotics in humidified incubator with 5% CO2 at 37 C. All sOMF exposures as well as control sets were carried out in a humidified incubator with 5% CO2 at 37 C. Dihydroethidium (Fisher Scientific) was dissolved in DMSO at 10 mM concentration (stored at −20 C) and diluted to 5 μM in culture media immediately before use.

### Static and Oscillating Magnetic Field Exposure

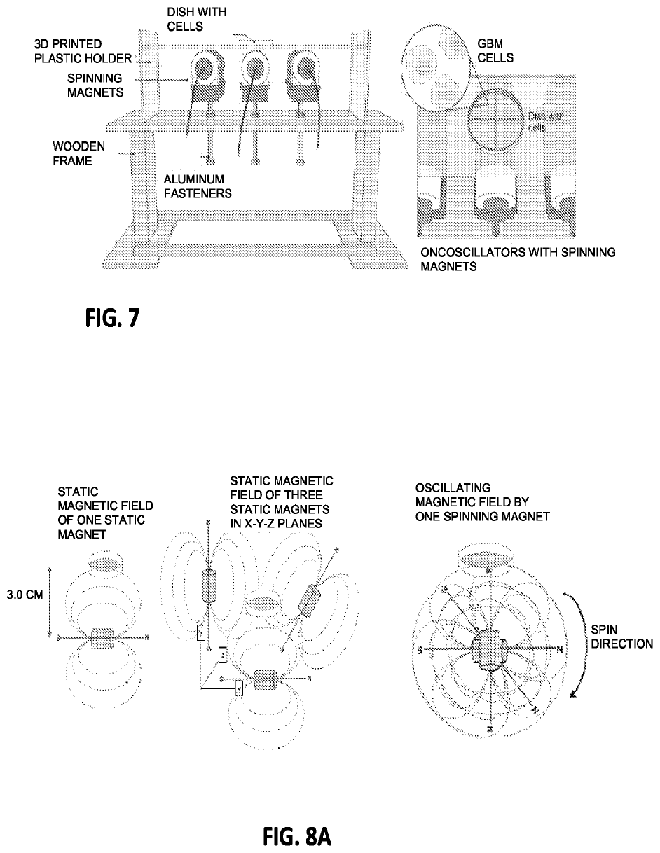

sOMF stimulation produced by spinning magnets was delivered using oncoscillators held by a specially constructed non-magnetic wooden frame with aluminum fasteners and 3D printed plastic holders, illustrated in FIG. 7. Oncoscillators were positioned at different distances from the cell culture plates to change the peak-to-peak amplitude (PPA) of the sOMF seen by the cells to ˜0.4, ˜1 or ˜5 mT. The values of magnetic flux density at the various distances from the axis of the axially magnetized cylindrical neodymium (N52) magnets used in the oncoscillators were measured using a Homend handheld digital WT10A gauss meter. Three different values of peak frequency (PF) of rotation of the magnet (sOMF oscillation frequency) were used—˜77 Hz, ˜137 Hz and ˜277 Hz. For intermittent stimulation, the on time (Tₒₙ or sOMF pulse train duration) was kept constant at 250 ms. The off times (T_{off}) were set at 250, 750 or 2750 ms.

All frequency and timing values were programmed into the microprocessor controlling the oncoscillators (see FIG. 1). sOMF effects produced by each set of parameters were compared with the other two sets and with unstimulated controls. Comparisons were also made with exposure to static magnetic field of one non-rotated magnet and three orthogonally oriented non-rotated magnets, as well as with the sOMF produced by rotating the latter three magnets. Stimulation with sOMF produced by a non-rotating Helmholtz coil was also carried out by passing a sinusoidal current of a strength sufficient to produce a PPA of ˜5 mT in between its two solenoids where the cell culture plate was placed. The sinusoidal current was generated by a function generator (Wavetek, San Diego, CA) and amplified to the desired current amplitude by a high current amplifier (Taidacent, Shenzhen Taida Century Technology Co., Ltd., China). Cells were stimulated once for a total duration of 4 h in each experiment. In one experiment they were stimulated for 2 h thrice with two 2-h intervals between the three stimulation periods to test for effects of repeated stimulations. Intermittent stimulation for 4 h was also compared with continuous stimulation for the same duration at the same PF and PPA.

### ROS Detection and Caspase 3 Activation Assay

Cells grown in glass bottom four-chamber dishes (one cell line in two chambers) were incubated with 5 μM dihydroethidium (hydroethidine) for 30 min in the humidified incubator with 5% CO₂ at 37° C. in dark. From each chamber 3-4 fluorescence images were randomly taken at every time point for each treatment group using Carl Zeiss microscope. Images before starting the static magnetic field or sOMF exposure (0 h), after 2 h and 4 h of sOMF exposure were captured. Last image was taken 2 h after ending the sOMF exposure (2 h-post). For caspase-3 activity detection, cells were incubated with NucView 488 dye for 30 min 12 h after 4 h of sOMF exposure. Cells were then fixed with 4% PFA, permeabilized with Triton-X 100, and incubated with DAPI for 10 minutes before imaged. Exposure time and magnification was kept same for all images in all experiments. Unmodified fluorescence micrographs, stained with hydroethidine at 10× resolution, were digitized on a desktop computer interfaced with the microscope and imported into the MATLAB programming environment (Mathworks, Natick, MA). MATLAB scripts written in house were used to automatically count RGB-encoded red cell pixels with fluorescence intensity above a uniform threshold setting. The intensity values obtained were normalized with respect to T0, i.e., pre-stimulation time point in each stimulation condition and the first time point in the no stimulation condition, or the average of all T0 values. The normalized value at each time point in the stimulation conditions was then renormalized with respect to the normalized value at the corresponding time point in the no stimulation condition. Data were pooled from three repetitions of each experiment and two-tailed Student's pooled t test with false discovery rate test for multiple comparisons was used to assess statistical significance atp=0.05 level.

### Clonogenic Cell Survival Assay

BT-115 or DIPG cells were seeded 200 cells per dish for clonogenic assay and kept in a humidified incubator with 5% CO₂ at 37° C. After 8-10 hours these dishes were transferred to an incubator in which the sOMF device was run. Following sOMF exposure dishes were transferred to cell culture incubator and were allowed to grow colonies for 10 days in case of DIPG cells and 14 days in case of BT-115 cells. Colonies were fixed and stained with crystal violet stain (0.05% crystal violet, 1% formaldehyde, 1% methanol) for 15-30 min. Washed and dried colonies were manually counted. Survival fraction was calculated by dividing number of colonies by number cells seeded and normalized with the average of survival fraction of control in respective experiment. The survival experiment was repeated, and average of experiments was plotted using Prism program in scatter with bars method. Each dot in the scatter represents normalized survival fraction of one dish.

Data were pooled from three repetitions of each experiment and two-tailed Student's pooled t test with false discovery rate test for multiple comparisons was used to assess statistical significance at p=0.05 level.

Effects of Static Magnetic Field Compared to sOMF

It was hypothesized that the interaction of weak and intermediate strength magnetic fields with the RPM mechanism in the mitochondrial ETC can perturb the electron transfer process (EMP hypothesis) to generate superoxide. The ETC membrane complex molecules are oriented in all directions and do not tumble, unlike molecules in solution. It was predicted, therefore, that a spinning magnet should induce more ROS than a non-rotating magnet oriented along one fixed axis (1Dst). Furthermore, the static fields of three non-rotating magnets oriented along the three orthogonal axes in 3D space (3Dst) should also generate ROS comparable in amount to the spinning magnet (1Dsp).

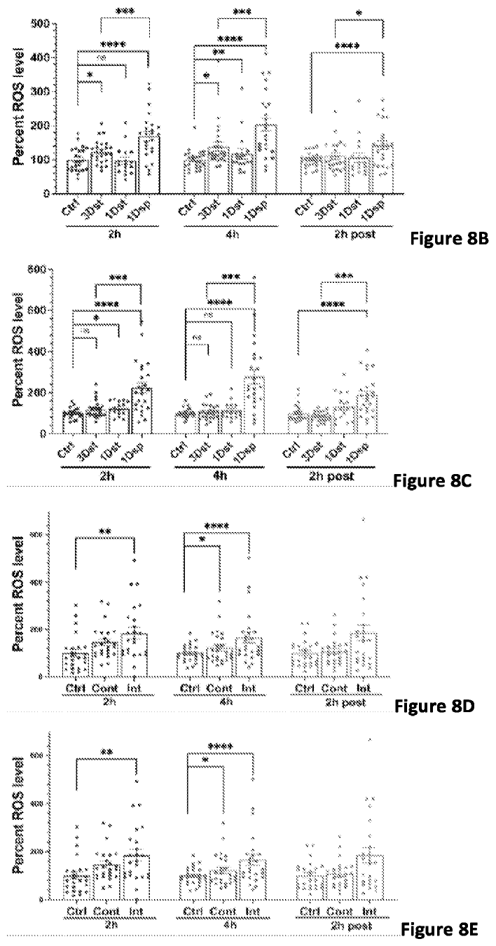

These predictions were tested by stimulating GBM and DIPG cells under all three conditions side by side (FIG. 8A). sOMF generated by an oncoscillator with a spinning magnet had a PF of ˜272 Hz and Tₒₙ and T_{off} of 250 ms each. The stimulation was carried out for 4 h. It was observed that in both GBM and DIPG cells ROS generated by sOMF (1Dsp) was significantly higher than that generated by both 1Dst and 3Dst static magnetic fields at 2 h (during stimulation), 4 h (at the end of stimulation) and 6 h (post-stimulation) (FIGS. 8C and 8C). While the increased effectiveness of a spinning magnetic field compared to a static field along one dimension confirms a prediction of the MEP hypothesis, the lack of a significant ROS-inducing effect of static fields oriented in all three dimensions suggests that the field oscillations themselves are important for this effect.

Comparison Between Intermittent and Continuous sOMF

If the total amount of exposure to oscillations at the peak frequency is important for ROS induction, a greater increase in ROS with continuous stimulation should be expected. Therefore, it was examined whether there is a difference between continuous and intermittent sOMF stimulation with oncoscillators. Intermittent stimulation was delivered with Tₒₙ and T_{off} of 250 ms each. The PPA for both types of stimulation was ˜277 Hz. The analysis shows that the level of ROS in cells exposed to continuous sOMF were not significantly higher from those exposed to intermittent sOMF in both GBM and DIPG cells. These data indicate that repeated pulse trains rising to and declining from the peak frequency with intervening pauses are sufficient to achieve near maximum level of increase in ROS.

Effect of sOMF Along One Fixed Axis

Magnetic field oscillations induced by a rotating magnet are characterized by two distinct components: the sinusoidal waves of the field and the cyclically changing angles of the axis of the magnetic field. The above results indicate that field oscillations play an important role in inducing ROS. To test whether oscillations are sufficient to produce the ROS effect, the effect of magnetic field oscillations produced by a Helmholtz coil, whose axis remains fixed in one orientation, was compared with those produced by the rotating magnet of an oncoscillator.

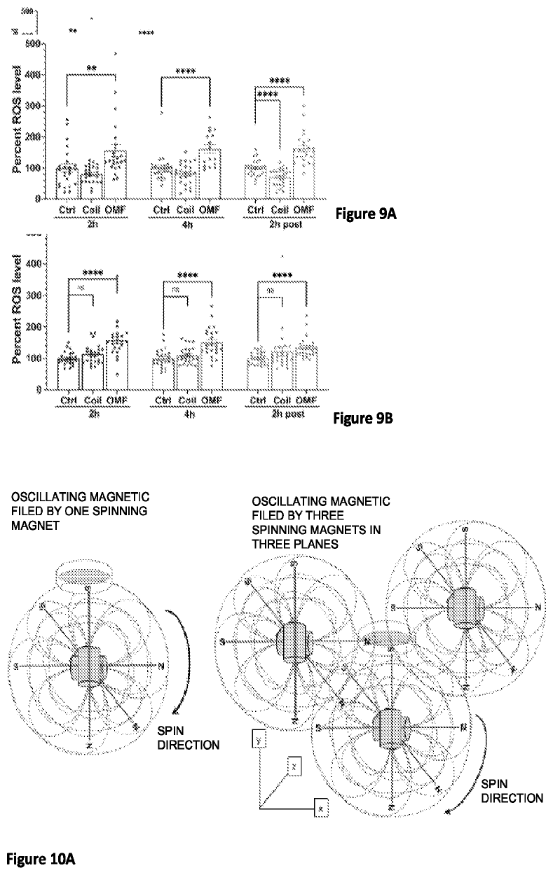

The current passing through the coil alternated at ˜137 Hz and was large enough to generate a PPA of ˜5 mT, values that were identical to those produced by the spinning magnet of an oncoscillator used in this experiment. Both apparatuses delivered continuous stimulation for 4 h. It can be seen that Helmholtz coil did not produce any significant increase in ROS at 2 and 4 h during stimulation or 2 h post-stimulation in GBM and DIPG cells (FIGS. 9A and 9B). In contrast, oncoscillator significantly raised the ROS levels at all three time points in both cancer cell types. These observations indicate that an oscillating field alone may not be sufficient to induce ROS, and that the changing angle of the magnetic axis may be required to achieve this effect.

### Higher ROS Levels Produced by Magnet Rotation Along Three Axes

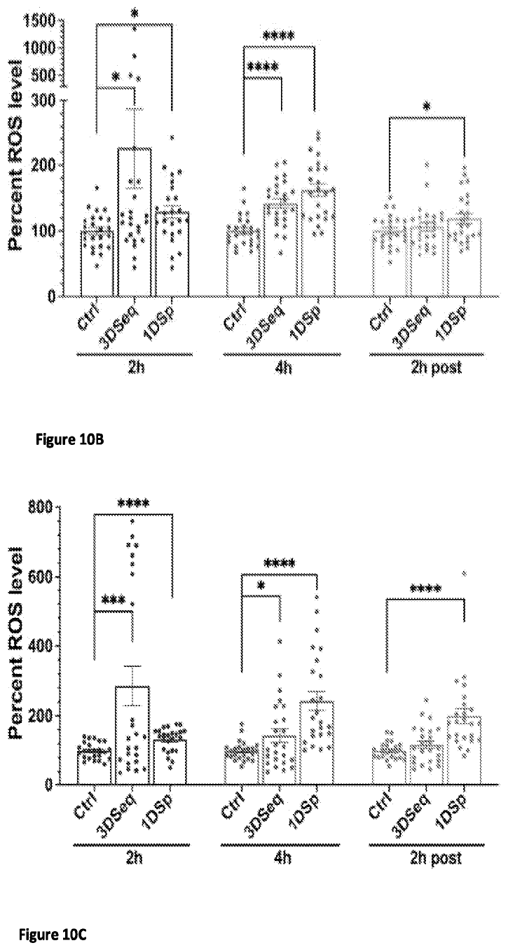

Because repeated changing of the angle of the magnetic field axis in all three dimensions may have a greater impact on ETC complexes oriented in all directions in space, it was further investigated whether rotating magnets along all three orthogonal axes in three-dimensional space potentiates further the increase in ROS produced by magnet rotation along only one axis. Three oncoscillators (3DSeq, FIG. 10A) were positioned at right angles to each other and were activated in repeating sequential or alternating cycles compared to intermittent stimulation with a single oncoscillator (1DSp). This experiment showed a greater increase in ROS at 2 h in both GBM and DIPG cells with 3DSeq compared to 1DSp stimulation; however, this difference was not statistically significant. The significant increases in ROS levels over control seen at 4 h and 2 h post-stimulation, are also not significantly different between 3DSeq and 1DSeq (FIGS. 10B and 10C). This suggests that 1DSp stimulation may be sufficient to produce maximal ROS enhancement given that one activated oncoscillator sweeps through all angles in a two-dimensional plane.

### Variation in Stimulus Parameters

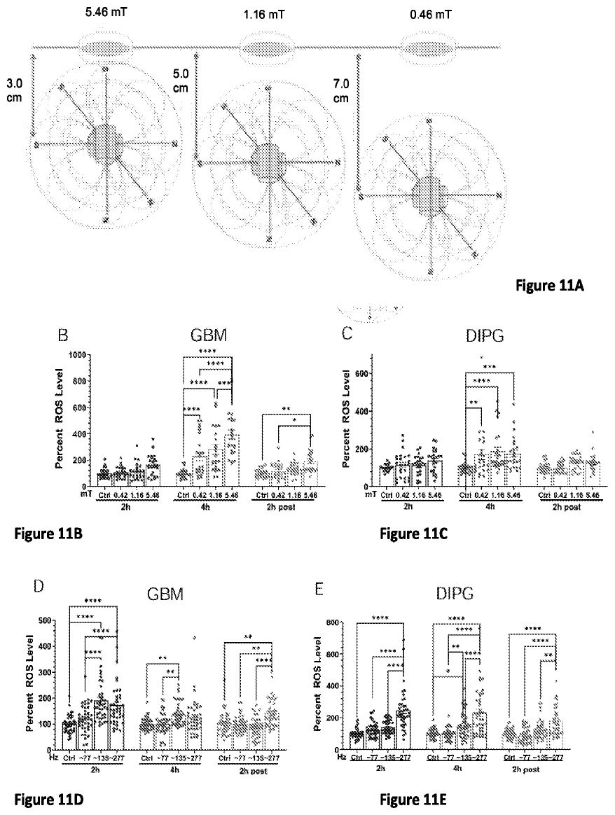

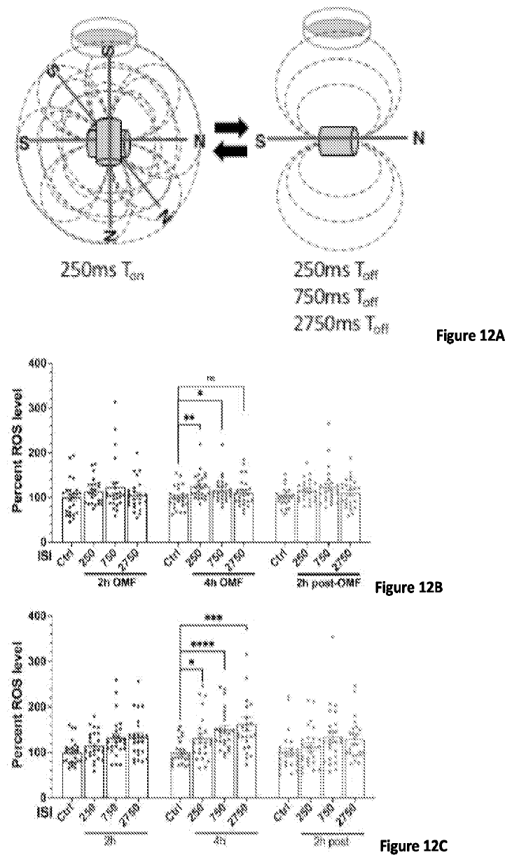

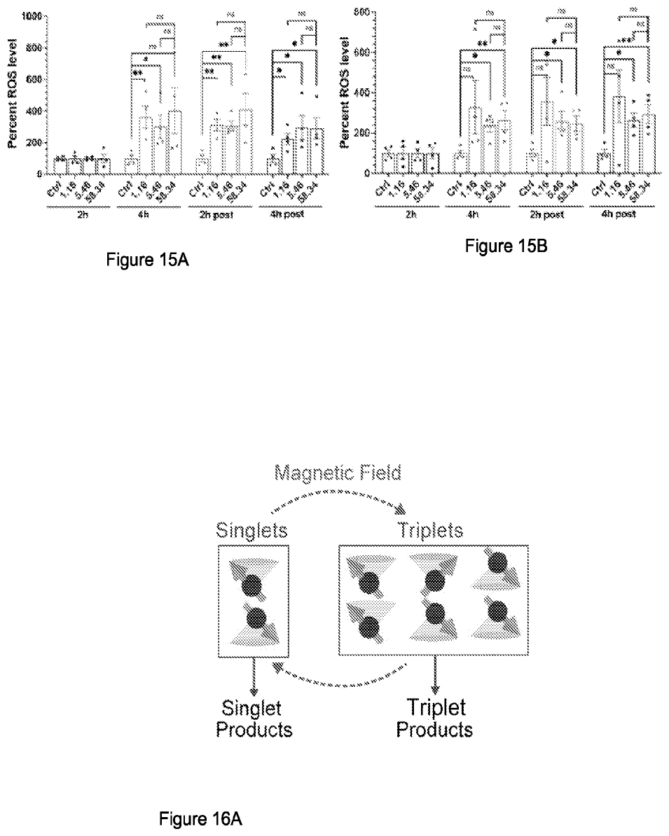

Stimulation with an oncoscillator has three physical parameters that can be varied along a continuous scale. The relevant parameters are (1) strength of the magnetic field or PPA of the stimulus, (2) the stimulus PF and (3) the T_{off} time. Tₒₙ was kept constant at 250 ms, and the effects of varying the values of each of the other parameters to three different levels were determined. The effect of ˜0.42 mT, ˜1.2 mT, ˜5.5 mT and 58.3 mT were studied by positioning the cell culture dishes at a distance of 7 cm, 5 cm, 3 cm and 1.4 cm respectively from the oncoscillator (FIG. 11A; see also FIGS. 15A and 15B). All field strengths tested showed significant increase in ROS levels at the 4 h time point in GBM and DIPG cells (FIGS. 11B and 11C). In terms of variation of PF between ˜77, ˜135 and ˜277 Hz, the latter two frequencies were significantly more effective than ˜77 Hz (FIGS. 11D and 11E). Comparing three different T_{off} values (250, 750 and 2750 ms) while keeping Tₒₙ 250 ms showed that in GBM cells 250 and 750 ms T_{off} produced slightly better effect than 2750 ms on ROS generation (FIGS. 12A and 12B). In contrast, in DIPG cells maximum ROS was generated by 2750 ms T_{off}(FIG. 12C).

### Effect of Repeated Stimulation

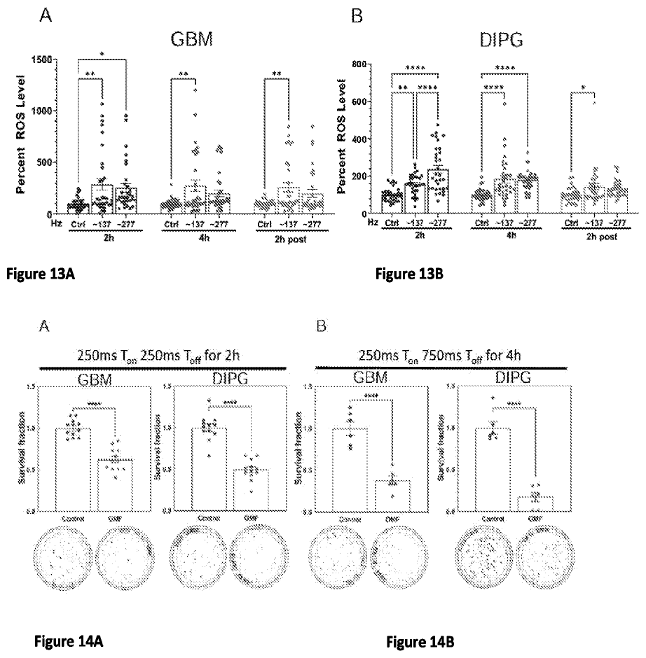

To determine whether repeated sOMF stimulation produces a cumulative increase or any other effect on ROS levels, cells were stimulated for 2 h thrice at 2-h intervals. This stimulation pattern using optimum values of PPA, T_{off} and PF produced similar increases in ROS levels in both GBM and DIPG cells at each subsequent repetition compared to the first stimulation session (FIGS. 13A and 13B). This suggests neither potentiation nor desensitization of the ROS inducing mechanism underlying the sOMF effect produced by a spinning magnet.

sOMF Effects on Cancer Cell Clonogenicity and Caspase 3 Activation

Investigating various parameters of stimulation allows one to identify the most effective range for the maximal induction of ROS in GBM and DIPG cells. Because higher ROS can cause oxidative damage to macromolecules and trigger cell death, the optimally effective parameters to stimulate GBM and DIPG cells and assess clonogenic cell survival and activation of caspase 3 were used. The parameters in the optimum range used were intermittent mode of stimulation with PPA of ˜5 mT, PF of ˜137 Hz, Tₒₙ of 250 ms, T_{off} of 750 ms and stimulation duration of 4 h. A standard clonogenic cell survival assay with the optimized parameters was conducted to find out whether and how much reduction in the survival fraction is caused by sOMF stimulation for 2 h and 4 h in the single cell condition. The 2-h stimulation had a T_{off} of 250 ms and 4-h of 740 ms. This corresponded to 28,800 and 19,200 pulse trains for 2-h and 4-h stimulations, respectively. With 2-h stimulation, an >60% and >40% reduction in DIPG and GBM survival fraction with sOMF, respectively (FIG. 14A), were observed. For 4-h stimulation, the respective values were >80% and >60% (FIG. 8B). This result is likely due to an immediate cytotoxic effect produced by short periods of sOMF stimulation on single cancer cells and a possible delayed cytostatic effect on them, in addition, to halt cell proliferation. Additionally, a modest increase in caspase 3 activation was observed in both DIPG and GBM cells 12 hours after the 4-h stimulation with optimum parameters.

### Potential Application of the Techniques

It was shown that first-in-human compassionate use monotherapy with the Oncomagnetic Device in a patient with end-stage recurrent GBM causes tumor size reduction with evidence of treatment effect in brain autopsy and the treatment is well tolerated. It was further shown that sOMF generated by oncoscillators are selectively cytotoxic to several cancer cell lines, including human GBM and U87 cells, A549 lung cancer cells and GL261 mouse GBM cells. Furthermore, it has been demonstrated that sOMF is not toxic to normal human astrocytes and cortical neurons in culture.

Here, the applicants tested the MEP hypothesis, stated above, and determined the effectiveness of several sets of sOMF stimulation parameters in inducing the superoxide component of ROS. The applicants further explored whether sOMF stimulation with an optimized set of parameters shows high anticancer potency in standardized assays. It was found that concurrent exposure to oscillations of a magnetic field produced by rotations of its axis in a two-dimensional plane cause a maximal rise in superoxide component of ROS within human GBM, human DIPG and GL261 mouse GBM cells.

A narrow range of magnet rotation frequencies (100-300 Hz) in the super low frequency domain, as defined by the International Telecommunications Union ⁴⁴, are effective in raising ROS to high levels. The strength of the field showing ROS-inducing efficacy is in the intermediate range of ˜1-˜5 mT. Increasing the strength to ˜58 mT does not significantly enhance the effect further, while lowering it to ˜0.4 mT shows a decreasing trend. Intermittent sOMF patterns do not appear significantly different from continuous sOMF stimulation in terms of their effectiveness. The combinations of on-off timing sequences chosen for intermittent stimulation show small differences in the observed effects. A set of values chosen for 2-h and 4-h session of stimulation from these optimum ranges of parameters are effective in killing GBM and DIPG cells and reducing the fraction of surviving colonies in a clonogenic cell survival assay. Even though the total number of pulse train stimuli of the same duration (250 ms) applied over 4 h (19,200) is less than that applied over 2 h (28,800), 4-h stimulation produces a more pronounced suppression of the growth of GBM and DIPG cell colonies. This indicates that the total amount of energy delivered to cancer cells is clearly not the determinant of the potency of stimulation. Instead, it appears that the longer T_{off} between stimuli of 750 ms in the 4-h stimulation, as opposed to 250 ms in the 2-h stimulation may be the critical variable. Alternatively, sustained stimulation over a longer period may be responsible for the stronger effect. Mitochondria are the predominant site of production of ROS, and the longer T_{off} may be important for the superoxide generated to trigger mitochondrial membrane permeability transition (MPT), although extra-mitochondrial sources such as cryptochromes cannot be ruled out. The biological effects cannot be accounted for by stimulation-induced hyperthermia because exposure to sOMF generated by the oncoscillators do not cause a rise in temperature at the distance separations between their magnets and the stimulated cell cultures used in these experiments.

It is noted that TTF treatment that shows an increase in H₂O₂ is reported to be 24 h in duration. Similarly, in contrast to >60% and >80% decreases in cell survival fraction in GBM and DIPG cells, respectively, with 4 h of sOMF stimulation by the device of this disclosure, TTF stimulation of 3-day duration at most produces a decrease of ˜10-˜50% in three different types of GBM cell lines. TTF also produces a paradoxical increase in clonogenic survival in the U251 GBM cell line. These differences indicate that underlying mechanisms of action between TTF and sOMF stimulations are distinct from each other.

Further, sOMF induction of ROS indicates that the immediate targets of its action are redox mechanisms in cells. Magnetic fields at or above 1 mT are known to alter the kinetics and yields of certain chemical reactions involving free radical intermediates exchanging unpaired electron. These effects are now recognized in the context of RPM to arise from the conservation of electron spins in radical recombination reactions and long-lasting spin coherences of spin-correlated electrons in radical pairs. RPM affected by magnetic flux density in the 1-10 mT range is characterized by: (1) Reactants that produce a pair of radicals with electron spins in a singlet configuration, i.e., with antiparallel spins; (2) Spin-selective reaction of singlet pairs to produce a singlet product or a non-selective back reaction; (3) Competition between these reactions and interconversion between singlet and triplet (parallel spins) pairs affected by an externally applied magnetic field; (4) The triplet pairs also reacting spin-selectively or back reacting non-selectively to give rise to triplet products; (5) In many instances, the non-selective product regenerating the initial reactants to restart the cycle; and (6) Singlet and triplet states affected by electron-nuclear hyperfine interactions.

In accordance with the MEP hypothesis, it is proposed that electron transfer processes in the ETC complexes may have these attributes, and therefore, may be perturbed optimally by sOMF in the observed ˜1-˜5 mT range (FIG. 10). The observation that there is no significant increase in the ROS induction effect at an order of magnitude increase in magnetic flux density (from ˜5 mT to ˜58 mT) is consistent with observations in the field of spin chemistry that the decrease in product yield in RPM reactions plateaus the flux density range of ˜1 mT—>1 T. The dependence of field oscillations and the orientation of the axis of the field together for the maximal induction of ROS in these experiments therefore supports the hypothesis that RPM is the target of sOMF. That rotation of a permanent magnet can achieve both makes this mode of magnetic stimulation particularly convenient. As an experimental confirmation of perturbation by sOMF of electron flow in the ETC, it has been shown separately with oxygen electrode measurements that sOMF halts or retards the consumption of oxygen by isolated rat liver mitochondria and permeabilized GBM and DIPG cell. It also has been observed that sOMF triggers mitochondrial MPT which is blocked by bongkrekic acid. Superoxide opens MPT pores in the inner mitochondrial membrane.

Thus, sinusoidal magnetic field oscillations combined with rapidly changing angles of the magnetic field axis in defined frequency and timing patterns at low mT flux densities can induce a rapid increase in the superoxide component of ROS in GBM and DIPG cells in culture, trigger their death and prevent growth of their colonies. The bridge between these biological effects and the physical influence of magnetic field appears to be quantum effects involving RPM underlying electron transfer reactions in the mitochondrial respiratory chain. The present experimental results provide a rational basis for employing sOMF produced by spinning permanent magnets in noninvasive treatment of GBM, DIPG and other solid malignancies.

### Impact of Oscillation Patterns on GBM and DIPG Cells

Thus, as illustrated in FIGS. 8A-E, sOMF of a spinning magnet generates higher cellular ROS than static magnetic field. FIGS. 9A and 9B illustrate the benefit of field oscillations and axis rotations for for ROS induction. FIGS. 10A and 10B illustrate that one spinning magnet or three spinning magnets generate similar cellular ROS levels. FIGS. 11A-E illustrate the effect of variation in magnetic field strength, peak frequency, and stimulation repetition. FIGS. 12A-C illustrate variation in the OFF period between stimulus trains. FIGS. 13A and 13B illustrate that sOMF with optimum parameters induces consistent increase in ROS in GBM and DIPG cells. FIGS. 14A and 14B illustrate that sOMF exposure causes significant cell death in GBM and DIPG cells. FIGS. 15A and 15B illustrate that increasing magnetic field strength from ˜5 mT to ˜58 mT appears to show no further ROS increase.

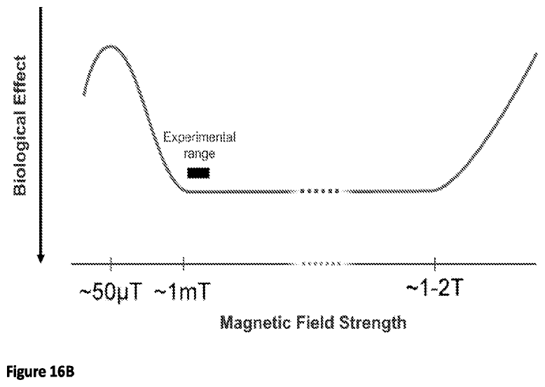

FIG. 16A illustrates magnetic field affecting the interconversion of electron spins from the singlet state to the triplet state. In particular, FIG. 16A shows vector representations of the spins. FIG. 16B illustrates that the effective range of the field strengths (˜1 mT-˜58 mT) used in the experiments described in this disclosure falls in the plateau phase of the biological effect. The arrow points in the increasing direction of the biological effect (i.e., increasing superoxide).

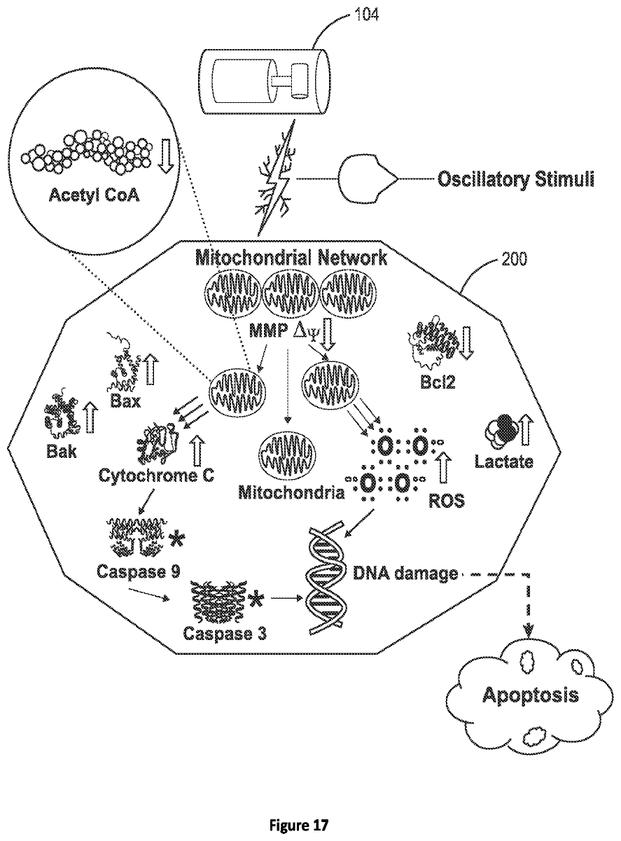

Finally, FIG. 17 schematically illustrates interaction between the system 100, here represented by an example microstimulator 104, and mitochondrial function of an example GBM cell 200. The microstimulator 104 (or several such microstimulators, as discussed above) generates OMFs that decrease mitochondrial glucose oxidation in the tricarboxylic acid (TCA) cycle (or “Krebs cycle”) via the production of [1,2-¹³C]acetyl-CoA, increase metabolic flux of glycolysis (determined via ¹³C enrichment of lactate from ¹H NMR spectrum of the cell extracts), disrupts electron flow in the ETC, increases superoxide, peroxide, and other reactive oxygen species generation, opens the MPTP and activates caspase-3-mediated or an alternate apoptotic pathway in GBM cells.

In particular, the system 100 causes one or more stimulators 104 to generate a rapidly changing magnetic field, which in turn disrupts electron flow in the cells 200 present in the tissue exposed to the magnetic field. As schematically illustrated in FIG. 2, the system 100 creates electrical perturbation of mitochondria, which are intracellular energy-producing components, and generates localized release of harmful chemicals (reactive oxygen species and cytochrome C) within cells with impaired mitochondrial function including, but not limited to, cancer cells. This localized release of harmful chemicals triggers apoptosis, or a molecular cell death process.

More particularly, cancer cells demand more energy in the form of adenosine triphosphate (ATP) produced by mitochondria due to uncontrolled cell divisions, and thus are under heightened stress. The system generates the OMF which in turn produces rapidly fluctuating or sustained depolarizations of the mitochondrial membrane potential (MMP) in the tissue. This process leads to fragmentation of mitochondrial networks and disruption of ATP generating proton flux/electron transport in individual mitochondria. Further, this process cases leakage of cytochrome C and reactive oxygen species (ROS) which depolarize the MMP further and cause degradation of mitochondria. These events collectively trigger the molecular pathway that leads to DNA damage and apoptosis in the cancer cells. Because normal cells (e.g., healthy cells) have a larger amount of mitochondria, have lower demand for ATP, and are not under stress, disruption of electron flow and small amount of ROS formation and MMP depolarization does not trigger apoptosis in normal cells. The lack of apoptosis may also be due to triggering of antioxidant mechanisms that counteract ROS increase.

During several tests discussed below, the system 100 in one implementation generated oscillating magnetic fields which, when applied to GBM cells, caused the breakdown of mitochondrial networks, disintegration of mitochondria due to MMP depolarization, and a decrease in Krebs cycle metabolites. The system 100 in this implementation included a single microstimulator with a neodymium magnet magnetized at 1.48 Tesla. The motor rotated the magnet at approximately 350 Hz. The system 100 generated stimulus pulses of approximately 500 ms duration, with an inter-stimulus interval of 1000 ms (as measured from the beginning of one pulse to the beginning of the next pulse). The microstimulator was placed at a distance of approximately 1 cm from the cells in a slide chamber. Intermittent stimulation with these 500 ms pulses, separated by 1000 ms, was conducted for 60 to 90 minutes.

In some embodiments, the microstimulators 104 oscillate at frequencies in the range of 250 to 350 HZ, to generate OMFs with frequencies of 250-350 Hz. In some cases, the frequencies at which the microstimulators 104 oscillate include subharmonic and superharmonic frequencies. The system 100 can apply OMFs as approximately 250 millisecond pulses with a 50 percent duty cycle (i.e., with an approximately 250 ms ON subcycle or pulse length followed by an approximately 250 ms OFF subcycle or inter-stimulus pulse interval), for example. The system 100 can nest these pulses (or other suitable pulses) in a supercycle that includes an ON period P_{ON} followed by an OFF period P_{OFF}. In some implementations, P_{ON} lasts between 5 and 900 seconds, and P_{OFF} lasts between 1 and 300 seconds. Additionally, the system 100 can ramp up the OMF frequency may be ramped up over a 75 to 100 millisecond period to a peak frequency, and subsequently ramped the OMF frequency down from the peak frequency over a 250 millisecond period.

Mitochondria in the experiments outlined below were stained with MitoTracker® and MitoSox® dyes. During repetitive stimulation and real-time imaging of live GBM cells over 10 and 90 minutes, and in fixed cells after 3 hours of stimulation, the fluorescence of these dyes changed, confirming the breakdown of mitochondrial networks, disintegration of mitochondria due to MMP depolarization, and a decrease in Krebs cycle metabolites due to the system 100 generating OMFs. Further, a decrease of approximately 10% in mitochondrial acetyl-CoA formation, combined with a similar increase in lactate (a byproduct of glycolysis in the cytoplasm) was observed using nuclear magnetic resonance spectroscopy.

The following list of examples reflects a variety of the embodiments explicitly contemplated by the present disclosure.

Example 1. A method for disrupting mitochondrial function in cells, the method comprising: causing, by controlling hardware, one or more magnets along multiple, substantially orthogonal, axis of rotation, to oscillate so as to generate an oscillating magnetic field; and applying the oscillating magnetic field to a tissue comprising cancer cells to trigger apoptosis in the cancer cells.

Example 2. The method of example 1, wherein a peak-to-peak amplitude (PPA) of the oscillating magnetic field is approximately 0.4 mT.

Example 3. The method of example 1, wherein a peak-to-peak amplitude (PPA) of the oscillating magnetic field is approximately 1 mT.

Example The method of example 1, wherein a peak-to-peak amplitude (PPA) of the oscillating magnetic field is approximately 5 mT.

Example 5. The method of example 1, wherein a peak frequency (PF) of rotation of the one or more magnets is approximately 77 Hz.

Example 6. The method of example 1, wherein a peak frequency (PF) of rotation of the one or more magnets is approximately 137 Hz.

Example 7. The method of example 1, wherein a peak frequency (PF) of rotation of the one or more magnets is approximately 277 Hz.

Example 8. The method of example 1, further comprising: generating, using the one or more magnets, intermittent stimulation, including generating a pulse train with an ON time of approximately 250 ms.

Example 9. The method of example 8, wherein the OFF time is approximately 250 ms.

Example 10. The method of example 8, wherein the OFF time is approximately 750 ms.

Example 11. The method of example 8, wherein the OFF time is approximately 2750 ms.

What is claimed is:

1. A method for disrupting mitochondrial function in cells, the method comprising:
causing, by controlling hardware, one or more magnets to oscillate along at least a first axis of rotation and a second axis of rotation substantially orthogonal to the first axis of rotation, so as to generate an oscillating magnetic field, including:
selecting the first axis of rotation,
applying an oscillation pattern along the first axis of rotation, the oscillating including a ramp-up period for an oscillation frequency, a ramp-down period for the oscillation frequency, and an inactive period following the ramp-down period, selecting the second axis of rotation, and
applying the oscillation pattern along the second axis of rotation; and
applying the oscillating magnetic field to a tissue comprising cancer cells to trigger apoptosis in the cancer cells.

2. The method of claim 1, wherein:
selecting the first axis of rotation includes selecting a first magnet in a first magnetic assembly; and
selecting the second axis of rotation includes selecting a second magnet in a second magnetic assembly.

3. The method of claim 2, including selecting the first axis of rotation and the second axis of rotation for concurrent operation.

4. The method of claim 2, including selecting the first axis of rotation and the second axis of rotation for sequential operation.

5. The method of claim 1, wherein:
selecting the first axis of rotation and the second axis of rotation includes a single magnetic assembly configured to change an axis of rotation of a magnet.

6. The method of any of the preceding claims, further comprising:
selecting a third axis of rotation orthogonal to the first axis and the second axis, and
applying the oscillation pattern along the second axis of rotation.

7. The method of any of the preceding claims, wherein the ramp-up period has a duration of 50 to 100 ms.

8. The method of any of the preceding claims, wherein the ramp-down period has a duration of 50 to 200 ms.

9. The method of any of the preceding claims, wherein the inactive period has a duration of 250 to 2750 ms.

10. The method of any of the preceding claims, wherein causing the one or more magnets to oscillate so as to generate the oscillating magnetic field includes:
causing the one or more magnets to reach a peak frequency of 50 to 300 Hz.

11. The method of any of the preceding claims, applying the oscillating magnetic field to the issue includes:
creating a peak-to-peak amplitude of the oscillating magnetic field in the tissue in a range of 1 to 200 mT.

12. The method of any of the preceding claims, including applying the oscillating magnetic field to the tissue for one to four hours per session, including repeating the session two to three time per day.

13. The method of any of the preceding claims, further comprising:
generating, during the ramp-up period and the ramp-down period, a pulse train with a duty cycle of 0.001% to 50%.

14. The method of claim 13, wherein the pulse train includes a square amplitude envelope.

15. The method of claim 13, wherein the pulse train includes a Gaussian amplitude envelope.

16. The method of claim 13, wherein the pulse train includes a sinusoidal amplitude envelope.

17. The method of claim 13, wherein the pulse train includes a ramp amplitude envelope.

18. The method of claim 13, wherein the pulse train includes a sawtooth amplitude envelope.

19. A device comprising:
at least one magnetic assembly configured to rotate a diametrically magnetized magnet around one or more axes; and
a controlling hardware configured to operate the at least one magnetic assembly according to a method of any of the preceding claims.
